(19) United States
(12) Patent Application Publication
(10) Pub. No.: US 2006/0141556 A1
(43) Pub. Date: Jun. 29, 2006

(54) Microwave cell lysis method using ionic additives

(76) Inventors: Sung-young Jeong, Gyeonggi-do (KR); Sang-hyun Peak, Seoul (KR)

Correspondence Address: CANTOR COLBURN, LLP, 55 GRIFFIN ROAD SOUTH, BLOOMFIELD, CT, 06002, US

(21) Appl. No.: 11/297,249

(22) Filed: Dec. 7, 2005

(30) Foreign Application Priority Data
Dec. 28, 2004 (KR) 10-2004-0113689

(51) Int. Cl. C12P 1/00; C12N 1/08

(52) U.S. Cl. 435/41; 435/270

(57) ABSTRACT

A cell lysis method in which an ionic additive and microwaves are used is provided. The cell lysis method includes: preparing a sample containing cells to be lysed; adding an ionic additive to the sample; and lysing cells in the sample by radiating microwaves onto the sample in which the ionic additive is added using a common microwave oven for a sufficient time to allow cell lysis to occur to a desired extent. According to the cell lysis method, even when a general microwave oven is used instead of expensive microwave equipment, regardless of the size of the sample, cell lysis can be rapidly performed by rapidly raising the temperature of the sample and a subsequent PCR process is not prohibited.

### BACKGROUND OF THE INVENTION

This application claims the benefit of Korean Patent Application No. 10-2004-0113689, filed on Dec. 28, 2004, in the Korean Intellectual Property Office, the disclosure of which is incorporated herein in its entirety by reference.

1. Field of the Invention

The present invention relates to a cell lysis method using microwaves, and more particularly, to a cell lysis method using heat by radiation of microwaves, in which ionic additives are used to obtain higher efficiency and reduce time and costs.

2. Description of the Related Art

Cell lysis is a process of releasing materials in a cell by disrupting a cell membrane and is generally performed to isolate DNA or RNA before amplification through a reaction such as a polymerase chain reaction (PCR).

Cell lysis is conventionally performed using a mechanical, chemical, thermal, electrical, ultrasonic or microwave method.

A chemical method includes the use of detergents, solvents or enzymes. Detergents disrupt a lipid double layer to release cell contents and lyse membrane protein. Detergents are most generally used to lyse animal cells and most denature protein. However, a reagent for cell lysis should be separately added, and hence a subsequent removing process is required, PCR inhibition occurs, and thus, the process takes a long time.

When using a thermal method, cell lysis can be performed by thermally contacting cells with a high temperature object, such as a hot plate. Such a thermal method usually requires a long time, and causes evaporation of moisture in a sample, thereby drying the sample. In addition, this method causes denaturation of protein, thus inhibiting the PCR.

Cell lysis can also be performed using ultrasonic treatment. This method includes placing a cell solution or suspension in a chamber located in an ultrasonic bath and performing ultrasonic treatment. However, such an ultrasonic disruption has many disadvantages in cell lysis. First, a nonuniform ultrasonic energy distribution causes inconsistent results. Second, the ultrasonic bath does not focus energy on the chamber and thus more time is required to complete cell disruption.

When using microwave radiation, cells are heated using microwaves to lyse the cells. Heating by means of microwaves can be performed more rapidly and efficiently performed than a conventional heating method, can be easily controlled by a switch, and does not result in excessive evaporation of a sample since the treatment time is short.

As described above, the cell lysis by microwave radiation meets the requirement that extraction of nucleic acids be rapidly and simply performed for rapid diagnosis. However, explosion in a container containing a sample may occur as a result of an increase in vapor pressure, and when the amount of the sample as little as it is equal to the wavelength of a microwave having a frequency of, for example, 2.45 GHz, cell lysis time increases, and is not efficient. In addition, when the amount of the sample is little, it is easily dried and adheres to a container wall, which often makes the use of the extracted nucleic acids in a subsequent step difficult.

There have been many attempts to solve these problems. In U.S. Pat. No. 6,623,945, the wavelength of the microwaves used was reduced by increasing the frequency of the microwaves to 20 GHz or greater so as to increase cell lysis efficiency even for small samples. However, since expensive microwave equipment must be used to increase the frequency, this method cannot be applied without the new equipment, and thus, practical utilization is difficult.

Thus, the inventors performed intensive research in order to overcome the above problems and discovered a cell lysis method which can be applied to small samples even when using conventional microwave equipment rather than expensive microwave equipment, has not danger of explosion since vapor pressure does not increase, rapidly achieves cell lysis due to a rapid increase in a sample temperature, and does not inhibit a subsequent PCR process.

### SUMMARY OF THE INVENTION

The present invention provides a cell lysis method in which an increase in vapor pressure can be prevented when microwaves are radiated onto a sample and the temperature of the sample can be increased rapidly and efficiently even at a low frequency by adding appropriate ionic additives to the sample when lysing cells with microwaves and extracting nucleic acids.

According to an aspect of the present invention, there is provided a cell lysis method including: preparing a sample containing cells to be lysed; adding an ionic additive to the sample; and lysing cells in the sample by radiating microwaves onto the sample in which the ionic additive is added using a common microwave oven for a sufficient time to allow cell lysis to occur to a desired extent.

In the method, the ionic additive may be a zwitterionic compound. The zwitterionic compound may be any compound including positive charge and negative charge in an equal amount and examples thereof includes CHAPS (3[(3-cholamidopropyl)dimethylammonio]-propanesulfonic acid), CAPS (3-(cyclohexylamino)-1-propanesulfonic acid), CAPSO (3-[cyclohexylamino]-2-hydroxy-1-propanesulfonic acid) or CHES (2(cyclohexylamino)ethanesulfonic acid). In the method, the ionic additive may be an ionic liquid. The ionic liquid is any ionic liquid known in the art and may be selected from compounds known as ionic liquids by Merck.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates the results of detecting a sample treated with microwaves after the addition of CHAPS and samples treated with microwaves without an additive; and

FIG. 2 illustrates the results of detecting samples treated with microwaves after the addition of various zwitterionic compounds.

### DETAILED DESCRIPTION OF THE INVENTION

Hereinafter, the present invention will be described in more detail.

A cell lysis method according to an embodiment of the present invention includes: preparing a sample containing cells to be lysed; adding an ionic additive to the sample; and lysing cells in the sample by radiating microwaves onto the sample in which the ionic additive is added using a common microwave oven for a sufficient time to allow cell lysis to occur to a desired extent.

The cells to be lysed are not particularly restricted and the sample containing cells is also not particularly restricted as long as it contains cells, regardless of their amount and type. The type of cell sample may be a person's saliva, blood, other samples including cells, or a cell suspension including microorganism strains. The amount of the sample is also not particularly restricted.

The ionic additive added to the sample may be a zwitterionic compound, an ionic liquid, or another compound having a charge (positive charge, negative charge, or positive charge and negative charge).

The zwitterionic compound is a compound having both a positive charge and a negative charge within a molecule. Such compounds have strong electrical attraction and hydrogen bond interactions between molecules. When microwaves are radiated onto the sample mixed with the zwitterionic compound, friction is maximized due to ion exchange and the temperature rapidly rises.

In the method, any zwitterionic compound having the characteristics described above may be added to the sample. The zwitterionic compound may be selected from the group consisting of CHAPS, CAPS, CAPSO and CHES.

The zwitterionic compound is not particularly restricted, but compounds inhibiting a PCR by, for example, being adsorbed by extracted DNA or preventing PCR amplification for other reasons when performing cell lysis by radiating microwaves onto a mixture of the zwitterionic compound and the sample cannot be used. For example, as described in examples below, Merquat 2001, Merquat 3331, Merquat 281, etc. are zwitterionic compounds which boil very rapidly, thereby improving the temperature increasing rate, but their use is not preferable. This is because, for example, Merquat 281 has about 50 cations and about 50 anions aligned in a row, and thus is easily adsorbed by DNA, which makes the subsequent PCR process difficult.

The ionic liquid may be an ionic crystal in a molten state. The ionic crystal is composed entirely of ions and can absorb microwaves with a high efficiency. In particular, since the ionic liquid is relatively inert and stable up to 200° C., an explosion due to a sudden increase in vapor pressure when radiating microwaves onto the sample can be prevented, and thus cell lysis by raising the temperature can be more stably performed.

The ionic liquid is not particularly restricted and possible ionic liquids are described in literature [Ionic Liquids (Mercks), Organic Synthesis Using Microwaves and Supported Reagents, R. S. Varma in “Microwaves in Organic Synthesis,” A. Loupy (Ed.), Wiley-VCH, Weinheim, Chapter 6, pp 181-218 (2002), Microwave Organic Synthesis, R. S. Varma in “McGraw-Hill Yearbook of Science and Technology 2002”, pp 223-225, McGraw-Hill, New York, N.Y. (2001); and An Expeditious Solvent-Free Route to Ionic Liquids Using Microwaves, R. S. Varma and V. V. Namboodiri: Chem. Commun., 643 (2001)]. In particular, examples of the ionic liquid include alkylpyridinium cations and dialkylimidazolium cations.

Other charged additives capable of maximizing the microwave effect and allowing the temperature of the sample to stably increase can also be used. A compound having positive charges, negative charges, or both can be used in the method. Such a compound is not particularly restricted, but compounds inhibiting a PCR by, for example, being adsorbed by extracted DNA or making PCR amplification impossible due to other reasons when performing cell lysis by radiating microwaves onto a mixture of the charged compound and the sample cannot be used. For example, as described in examples below, SDS boils very rapidly, thereby increasing the temperature increasing rate, and effectively causes cell lysis, but its use is not preferable. This is because when adding SDS to a sample, it denatures cell protein due to its property as a detergent and inhibits a PCR by blocking the formation of a PCR band, which occurs because a PCR must be performed using extracted nucleic acids.

The amount of the ionic additive added is not critical in the present invention and can be appropriately selected according to the type of cell to be lysed, the concentration of the sample and the type of the ionic additive used to perform cell lysis through experiment. However, it is important to select an amount that causes no PCR inhibition, which may be easily recognized by those skilled in the art. For example, when a blood sample or a saliva sample is used and a zwitterionic compound such as CHAPS is used as the ionic additive, the amount of the ionic additive with respect to the amount of the sample can be appropriately selected by those skilled in the art according to the type of cell sample and the desired degree of cell lysis.

According to the method of the present embodiment, cells in the sample are lysed by radiating microwaves onto the ionic additive added sample using a general microwave oven for a sufficient time to cause cell lysis to the desired extent.

In the method, the microwave radiation is performed using a general microwave oven. All general microwave ovens currently use a standard frequency of about 2.45 GHz. One effect of the present invention is that cell lysis can be efficiently performed on all cell samples regardless of their types and amounts without separate expensive microwave equipment.

That is, the frequency of all general microwave ovens is standardized at about 2.45 GHz. Conventionally, when microwaves having said frequency are used, the time required to raise the temperature of a small sample is too long. For example, in the case of a blood sample, etc., since the amount of the sample used to isolate nucleic acids is small, rapid and efficient cell lysis cannot be performed. To solve these problems, as discussed above, U.S. Pat. No. 6,623,945 proposed the use of microwave equipment having a frequency ranging from 18 to 26 GHz, but the present invention enables an efficient temperature increase by using the ionic additive without using expensive equipment.

According to the method of the present invention, cell lysis efficiency is increased by rapidly raising the temperature of cells even at a low frequency, regardless of the size of the cell sample. Further, an excessive increase in vapor pressure is prevented by shortening the time required to reach the desired cell lysis temperature, and thus, even at high temperatures, the sample is stable.

As described above, according to the method of the present invention, cell lysis can be significantly facilitated by adding the ionic additive to the sample and radiating microwaves onto the sample. The inventors investigated whether such effects are due to chemical cell lysis resulting from the ionic additive itself in addition to the thermal cell lysis or are a synergism of them (Example 2). As a result, it was found that the detergent alone can cause little or no cell lysis in a short time of 1 minute or less, whereas this is the time required for cell lysis to occur according to the present invention.

Thus, the ionic additive in the present invention does not generate cell lysis by itself but plays an important role in synergic effect maximizing the cell lysis obtained through microwaves.

The present invention will now be described in greater detail with reference to the following examples. The following examples are for illustrative purposes only and are not intended to limit the scope of the invention.

### EXAMPLES

### Example 1

Measurement of time elapsed before boiling according to type of additive

Various additives were added to an E. coli cell sample and microwaves were radiated onto the sample to measure the time until the sample boiled. This measurement was carried out using the following method.

An E. coli suspension (3 ml) was incubated to exponential phase in a LB medium at 37° C. for 24 hours under aerobic conditions. The bacterial cells were collected by centrifuging and twice washed with 5 ml of a phosphate-buffered saline (PBS). The cells were resuspended in PBS.

0.2 g of ionic additives were added to 2 ml of the cell suspension. The mixture was placed on a general microwave oven and microwaves were radiated to measure the time until boiling was observed with the naked eye.

When observing the boiled cell sample after centrifuging, it was confirmed with the naked eye that no cell pellet was formed and a significant number of the cells were disrupted.

The times until samples to which various additives were added boiled is given in Table 1.

TABLE 1

| Additive | Time until boiling |
|---|---|
| Distilled water (Gibco) | 5:20 |
| Triton X-100 (nonionic) (Sigma) | 6:00 |
| Merquat 2001 (zwitterionic) (ONDEO Nalco) | 1:00 |
| Merquat 3331 (zwitterionic) (ONDEO Nalco) | 1:16 |
| Merquat 281 (zwitterionic) (ONDEO Nalco) | 0:20 |
| SDS (anionic) (Sigma) | 0:09 |
| Small bead (Sigma) | 2:30 |
| Large bead (Sigma) | 2:30 |
| Sand (Sigma) | 2:30 |
| CHAPS (zwitterionic) (Aldrich) | 0:26 |
| CAPS (zwitterionic) (Aldrich) | 0:26 |
| CAPSO (zwitterionic) (Aldrich) | 0:30 |
| CHES (zwitterionic) (Aldrich) | 0:30 |

The results of Table 1 can be illustrated as follows.

When distilled water was used as the additive and the sample was heated with a general microwave oven, the temperature increased very slowly. When Triton X-100, which is a nonionic detergent, was added and the microwaves were radiated, the temperature increased very slowly as well. It can be seen from these results that water and nonionic detergents do not affect the cell lysis caused by microwaves, and thus, effective cell lysis for the sample to which water or nonionic detergent is added cannot be achieved using a general microwave oven.

Referring to Table 1, when zwitterionic compounds, i.e., Merquat 2001, Merquat 3331, Merquat 281, CHAPS, CAPS, CAPSO, and CHES were used as the additive and the samples were heated with the general microwave oven, the increase in temperature occurred very rapidly. That is, the temperature increased faster than when small beads, large beads and sand capable of effecting an efficient increase in temperature were used. However, Merquat 2001, Merquat 3331, and Merquat 281 adsorb isolated DNA, and thus cannot be used in the subsequent PCR process. Although the zwitterionic compound increases the cell lysis by microwaves, if it inhibits a PCR, it cannot be used as the ionic additive of the present invention. This will be easily understood by those skilled in the art.

Among the above zwitterionic compounds, CHAPS, CAPS, CAPSO and CHES significantly reduced the time until boiling and did not inhibit the subsequent PCR process. Thus, these compounds did not need to be separately removed.

Referring to Table 1, when SDS was added as a charged compound, the increase in temperature was very rapid. However, the SDS prevented the formation of a PCR band in the subsequent PCR process as well. As indicated above, charged compounds which do not inhibit a PCR should be used, which will be easily understood by those skilled in the art.

### Comparative Example 1

A comparative experiment without microwave radiation was carried out as follows.

The above-described additives were added in equal amounts to a cell suspension prepared in the same manner as in Example 1 under the same conditions and let alone for 30 seconds without microwave radiation. Then, whether cell lysis occurred was observed.

Whether cell lysis occurred was observed with the naked eye as described in Example 1. It could be seen that when the microwave radiation was not used, cell pellets were clearly formed, indicating that cell lysis rarely occurred.

Thus, it was confirmed that the cell lysis of Example 1 was not caused by chemical lysis through the zwitterionic compounds such as CHAPS, CAPS, CAPSO, and CHES.

### Example 2

Cell lysis in a human's saliva was tested as follows.

CHAPS, CAPS and CHES were respectively added to 2 ml of saliva in a concentration of 10% and heated in a microwave oven until boiling to perform cell lysis. As a control, saliva without said compounds was used to perform cell lysis as described above.

To identify the cell lysis results, 1 μl of the cell lysed sample was taken and a PCR was performed. The PCR conditions were as follows.

Primers labeled with cy3 (primer 0.68 μM), 0.2 mM dNTP, a 2× PCR buffer (Solgent, Korea), and 2.5μ Taq DNA polymerase (Solgent, Korea) were reacted. The PCR was performed by predenaturing at 95° C. for 1 minute, performing 25 cycles (denaturation at 95° C. for 5 seconds, annealing at 60° C. for 13 seconds, and extension at 72° C. for 15 seconds) and performing an additional extension at 72° C. for 1 minute. The PCR product was inspected on a 3.5% agarose gel. However, the cell lysis results were not apparent on the agarose gel. Thus, a bacterial chip fabricated as follows was used to identify the cell lysis results.

Fabrication of bacterial chip

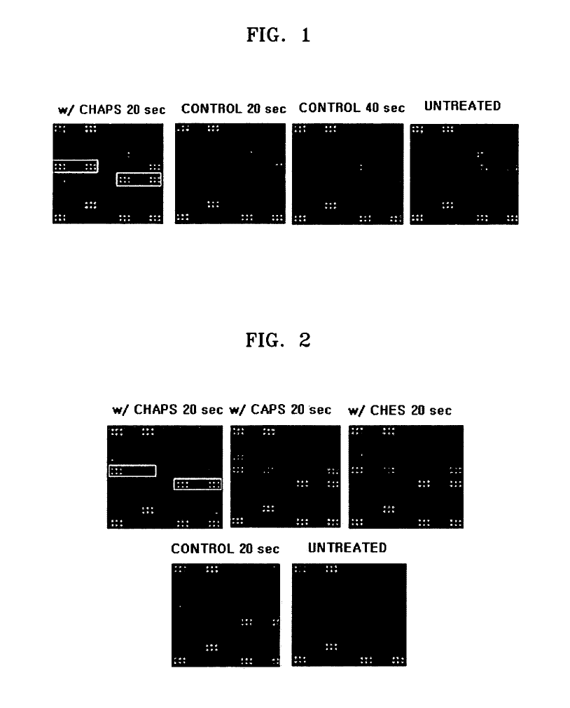

10 types of bacteria including Streptococcus pneumoniae, Heamophilus influenzae, Staphylococcus aureus, Pseudomonas aeruginosa, Legionella pneumoniae, Chlamydia pneumoniae, Mycoplasma pneumoniae, Bordetella pertussis, Klebsiella pneumoniae, and Moraxella catarrhalis as microorganisms which can be present in a human's saliva were selected as respiratory infective diseases, and probes and primers which can assay each species were selected to fabricate chips. 16S rRNA of each bacteria was searched for a portion having homology among invariable sites through multiple alignment, and then a universal primer for the portion was designed to prepare amplicon with 200-300 bp. A species specific probe for a variable site in the amplicon was designed. The bacterial chip having the above features was used to detect the cell lysis results for the saliva. Hybridization was performed for 30 minutes using a 6× SSPET buffer and a 3×-1× SSPET washing buffer at a temperature of 40° C. The results were obtained using an Axon Scanner and are illustrated in FIGS. 1 and 2.

FIG. 1 illustrates the results of detecting a sample including CHAPS and treated with microwaves and samples including no additive and treated with microwaves. The detection results of FIG. 1 are indicated in Table 2.

TABLE 2

| | CHAPS | Control | Control | |
|---|---|---|---|---|
| | (20 sec) | (20 sec) | (40 sec) | Untreated |
| HI_F | 9806 | 240 | 885 | 90 |
| HI_R | 2762 | 190 | 527 | 96 |
| SP_F | 7334 | 230 | 640 | 96 |
| SP_R | 5130 | 290 | 1422 | 131 |

Referring to Table 2, when adding CHAPS and treating with microwaves, cell lysis efficiency is 40 times higher than when treating with microwaves without an additive. Further, when adding CHAPS and treating with microwaves, the sample boiled after only 20 seconds and the viscosity of the saliva was significantly reduced.

FIG. 2 illustrates the results of detecting cell lysis in a sample treated with microwaves after the addition of various zwitterionic compounds. The results of FIG. 2 are indicated in Table 3.

TABLE 3

| | CHAPS | CAPS | CHES | Control | Untreated |
|---|---|---|---|---|---|
| HI_F2 | 1971 | 2980 | 3236 | 395 | 99 |
| HI_R2 | 315 | 396 | 591 | 227 | 98 |
| SP_F2 | 4079 | 9840 | 8460 | 1785 | 93 |
| SP_R2 | 4147 | 6302 | 7229 | 2483 | 100 |

Referring to Table 3, when various zwitterionic compounds were added and cell lysis was performed using microwaves, the sample boiled after only 20 seconds and the viscosity of the saliva was significantly reduced. In addition, the cell lysis efficiency was 10 times higher than for the control group.

In the cell lysis method using additives according to the present invention, even when a general microwave oven rather than expensive microwave equipment is used, regardless of the size of the sample used, there is no danger of an explosion since vapor pressure does not increase when raising the temperature, cell lysis can be rapidly achieved due to a rapid increase in the temperature of a sample, and chemicals do not need to be removed, and thus cell lysis does not inhibit a subsequent PCR process.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A cell lysis method comprising:
preparing a sample containing cells to be lysed;
adding an ionic additive to the sample; and
lysing cells in the sample by radiating microwaves onto the sample in which the ionic additive is added using a common microwave oven for a sufficient time to allow cell lysis to occur to a desired extent.

2. The cell lysis method of claim 1, wherein the ionic additive is a zwitterionic compound.

3. The cell lysis method of claim 2, wherein the zwitterionic compound is selected from the group consisting of CHAPS (3[(3-cholamidopropyl )dimethylammonio]-propanesulfonic acid), CAPS (3-(cyclohexylamino)-1-propanesulfonic acid), CAPSO (3-[cyclohexylamino]-2-hydroxy-1-propanesulfonic acid) and CHES (2(cyclohexylamino)ethanesulfonic acid).

4. The cell lysis method of claim 1, wherein the ionic additive is an ionic liquid.

5. The cell lysis method of claim 4, wherein the ionic liquid is at least one cation selected from the group consisting of an alkylpyridinium cation and a dialkylimidazolium cation.
